(19) United States
(12) Patent Application Publication
(10) Pub. No.: US 2006/0281767 A1
(43) Pub. Date: Dec. 14, 2006

(54) Triazoloypyrimidines

(75) Inventors: Olaf Gebauer, Koln (DE); Ulrich Heinemann, Leichingen (DE); Jorg Nico Greul, Leichlingen (DE); Stefan Herrmann, Langenfeld (DE); Oliver Guth, Leverkusen (DE); Hans-Ludwig Elbe, Wuppertal (DE); Herbert Gayer, Monheim (DE); Stefan Hillebrand, Neuss (DE); Ulrike Wachendorff-Neumann, Neuwied (DE); Karl-Heinz Kuck, Langenfeld (DE); Peter Dahmen, Neuss (DE)

Correspondence Address: STERNE, KESSLER, GOLDSTEIN & FOX PLLC, 1100 NEW YORK AVENUE, N.W., WASHINGTON, DC, 20005, US

(73) Assignee: BAYER CROPSCIENCE AKTIENGESELLSCHAFT, Moneheim (DE)

(21) Appl. No.: 10/561,174

(22) PCT Filed: Jun. 14, 2004
(86) PCT No.: PCT/EP04/06369

§ 371 Date: Jun. 6, 2006

(30) Foreign Application Priority Data
Jun. 24, 2003 (DE) 10328173.8

(51) Int. Cl. A01N 43/90; A61K 31/519; C07D 487/04

(52) U.S. Cl. 514/259.31; 544/256

(57) ABSTRACT

The invention relates to new triazolopyrimidines of the formula 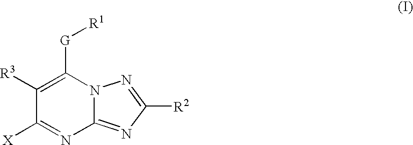 in which
R¹, R², R³, G and X have the meanings given in the description, a process for preparing these substances and their use for combating undesirable micro-organisms.
   The invention further relates to new intermediate products of the formulae 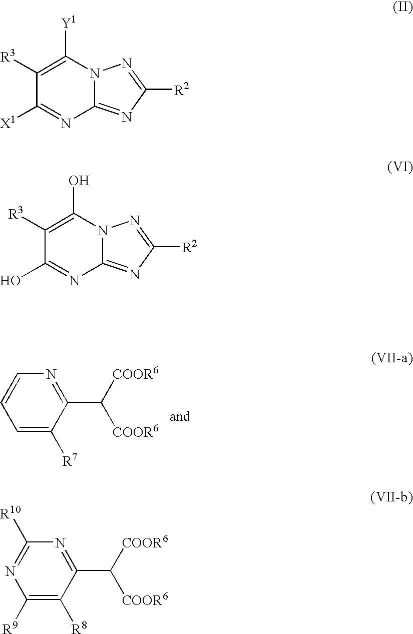 and processes for preparing these substances.

The present invention relates to new triazolopyrimidines, a method for their preparation and their use for combating undesirable micro-organisms. The invention also relates to new intermediate products and processes for their preparation.

It is already known that certain triazolopyrimidines possess fungicidal properties (cf. FR-A 2 784 380 and U.S. Pat. No. 6,277,857). The efficiency of these substances is good but in some cases, leaves something to be desired when low quantities are used.

New triazolopyrimidines of the formula 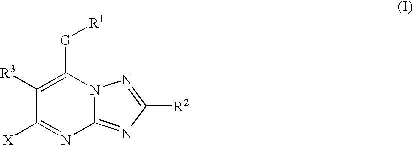
in which
R¹ represents optionally substituted alkyl, optionally substituted alkenyl, optionally substituted alkynyl, optionally substituted cyloalkyl or optionally substituted heterocyclyl,
R² represents hydrogen, halogen, optionally substituted alkyl or optionally substituted cycloalkyl,
R³ represents optionally substituted heterocyclyl
G represents oxygen or SOₙ, wherein
   n is 0, 1 or 2,
and
X represents halogen, cyano, optionally substituted alkyl, optionally substituted alkoxy, optionally substituted alkylthio, optionally substituted alkylsulphinyl or optionally substituted alkylsulphonyl,
have been found.

It has been further found that triazolopyrimidines of formula (I) can be prepared by reacting

(a) dihalogentriazolopyrimidines of the formula 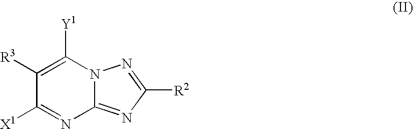
in which
R² and R³ have the meanings specified above,
X¹ represents halogen and
Y¹ represents halogen,
with compounds of the formula
   R¹-G-H (III)
   in which
R¹ and G have the meanings specified above,
optionally in the presence of a diluent, optionally in the presence of an acid acceptor and optionally in the presence of a catalyst and optionally triazolopyrimidines thus obtained of the formula 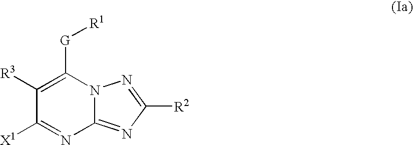
   in which
R¹, R², R³, G and X¹ have the meanings specified above,
either
α) with compounds of the formula
   R⁴-Me (IV)
   in which
   R⁴ represents optionally substituted alkoxy, optionally substituted alkylthio, optionally substituted alkylsulphinyl, optionally substituted alkylsulphonyl or cyano,
   Me represents sodium or potassium,
   optionally in the presence of a diluent,
   or
β) with Grignard compounds of the formula
   R⁵—MgHal (V)
   in which
   R⁵ represents optionally substituted alkyl and
   Hal represents chlorine or bromine,
   in the presence of a diluent.

Finally, it has been found that triazolopyrimidines of formula (I) are very well suited for combating undesirable micro-organisms. They primarily exhibit a strong fungicidal efficiency and can be used both for protecting plants and for protecting materials.

The triazolopyrimidines of formula (I) surprisingly have a substantially better microbicidal efficiency than the previously known substances having the same direction of action and the most similar constitution.

The compounds of formula (I) according to the invention can optionally be present as mixtures of various possible isomeric forms, especially of stereoisomers such as E, Z, threo and erythro isomers as well as optical isomers such as R and S isomers or atropisomers, but optionally also as tautomers.

Both the pure stereoisomers and also any mixtures of these isomers are the subject matter of this invention, even if in general only compounds of formula (I) are mentioned here.

According to the type of substituents defined above, the compounds of formula (I) have acid or basic properties and can form salts. If the compounds of formula (I) bear hydroxy, carboxy or other groups which induce acid properties, these compounds can be reacted with bases to form salts.

Suitable bases, for example, are hydroxides, carbonates, hydrogen carbonates of alkali and alkaline-earth metals, especially those of sodium, potassium, magnesium and calcium, furthermore ammonia, primary, secondary and tertiary amines with (C₁-C₄) alkyl residues as well as mono-, di- and trialkanol amines of (C₁-C₄) alkanols. If the compounds of formula (I) bear amino, alkylamino or other groups which induce basic properties, these compounds can be reacts with acids to form salts. Suitable acids are, for example, mineral acids such as hydrochloric, sulphuric and phosphoric acid, organic acids such as acetic acid or oxalic acid and acid salts such as NaHSO₄ and KHSO₄. The salts thus obtained likewise show fungicidal and microbicidal properties.

The subject matter of the invention also comprises salt-like derivatives formed from compounds of formula (I) by reaction with basic or acid compounds as well as N-oxide, which can be prepared by conventional oxygenation methods.

In the present case, heterocyclyl represents saturated or unsaturated, aromatic or non-aromatic, cyclic compounds with 3 to 8 ring members in which at least one ring member is a heteroatom, that is an atom other than carbon. If the ring contains several heteroatoms, these can be the same or different. Heteroatoms are preferably oxygen, nitrogen or sulphur. If the ring contains several oxygen atoms, these are not directly adjacent. The cyclic compounds optionally form a polycyclic ring system with further carbocyclic or heterocyclic, condensed or bridged rings. Mono- or bi-cyclic ring systems are preferred, especially mono- or bicyclic aromatic ring systems.

The triazolopyrimidines according to the invention are generally defined by formula (I). Preferred are those substances of formula (I) in which
R¹ represents alkyl with 1 to 6 carbon atoms, which can be identically or differently substituted between one and five times, by halogen, cyano, hydroxy, alkoxy with 1 to 4 carbon atoms and/or cycloalkyl with 3 to 6 carbon atoms, or
R¹ represents alkenyl with 2 to 6 carbon atoms, which can be identically or differently substituted between one and three times, by halogen, cyano, hydroxy, alkoxy with 1 to 4 carbon atoms and/or cycloalkyl with 3 to 6 carbon atoms, or
R¹ represents alkynyl with 3 to 6 carbon atoms which can be identically or differently substituted between one and three times, by halogen, cyano, hydroxy, alkoxy with 1 to 4 carbon atoms and/or cycloalkyl with 3 to 6 carbon atoms, or
R¹ represents cycloalkyl with 1 to 6 carbon atoms which can be identically or differently substituted between one and three times, by halogen and/or alkyl with 1 to 4 carbon atoms, or
R¹ represents saturated or unsaturated heterocyclyl with 5 or 6 ring members and 1 to 3 heteroatoms such as nitrogen, oxygen and/or sulphur, wherein the heterocyclyl can be substituted once or twice by halogen, alkyl with 1 to 4 carbon atoms, cyano and/or cycloalkyl with 3 to 6 carbon atoms,
R² represents hydrogen, fluorine, chlorine, bromine, iodine, alkyl with 1 to 4 carbon atoms, haloalkyl with 1 to 4 carbon atoms and 1 to 9 halogen atoms or cycloalkyl with 3 to 6 carbon atoms,
R³ represents saturated or unsaturated heterocyclyl with 5 or 6 ring members and 1 to 4 heteroatoms such as nitrogen, oxygen and/or sulphur, wherein the heterocyclyl can be identically or differently substituted between one and four times by
fluorine, chlorine, bromine, cyano, nitro, alkyl, alkoxy, hydroximinoalkyl or alkoximinoalkyl with respectively 1 to 3 carbon atoms per part alkyl,
haloalkyl or haloalkoxy with respectively 1 to 3 carbon atoms and 1 to 7 halogen atoms
G represents oxygen or SOₙ, wherein
   n is 0, 1 or 2,
   and
x represents fluorine, chlorine, bromine, cyano, alkyl with 1 to 4 carbon atoms, alkoxy with 1 to 4 carbon atoms, alkyl sulphinyl with 1 to 4 carbon atoms or alkyl sulphonyl with 1 to 4 carbon atoms.

Especially preferred are those triazolopyrimidines of formula (I), in which
R¹ represents a residue of the formula 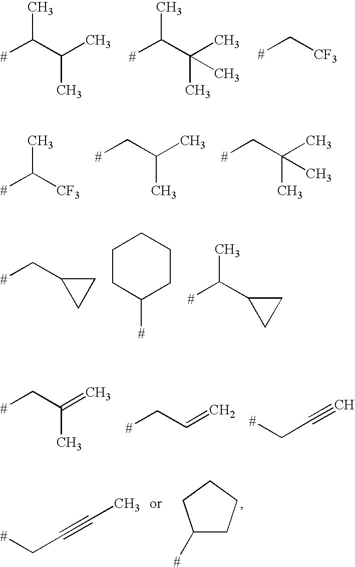
   where # marks the linking point,
R² represents hydrogen, fluorine, chlorine, bromine, iodine, methyl, ethyl, isopropyl, cyclopropyl, cyclobutyl, cyclopentyl, cyclohexyl, trifluoromethyl, 1-trifluoromethyl-2,2,2-trifluoroethyl or heptafluoroisopropyl,
R³ represents pyridyl which is linked in the 2- or 4-position and can be identically or differently substituted between one and four times by fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, or
R³ represents pyrimidyl which is linked in the 2- or 4-position and can be identically or differently substituted between one and three times by fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, or
R³ represents thienyl which is linked in the 2- or 3-position and can be identically or differently substituted between one and three times by fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, or
R³ represents thiazolyl which is linked in the 2-, 4- or 5-position and can be identically or differently substituted once or twice by fluorine, chlorine, bromine, cyano, nitro, methyl, ethyl, methoxy, methylthio, hydroximinomethyl, hydroximinoethyl, methoximinomethyl, methoximinoethyl and/or trifluoromethyl,
G represents oxygen or sulphur and
X represents fluorine, chlorine, bromine, cyano, methyl, methoxy or methylthio.

The aforementioned residue definitions can be arbitrarily combined amongst one another. In addition, individual definitions can be omitted.

If 5,7-dichloro-6-(5-chloro-pyrimidin-4-yl)-[1,2,4]-triazolo[1,5-a]pyrimidine and 1,2-dimethyl-propanthiol are used as initial substances, the course of the first stage of the method (a) according to the invention can be illustrated by the following formula scheme.

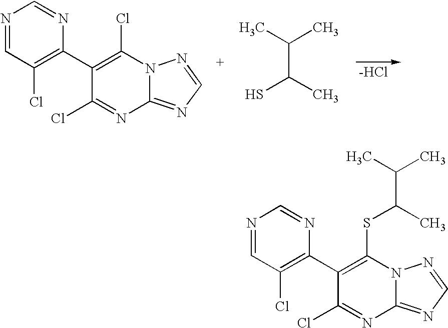

If the aforementioned compound prepared according to the first step of the method (a) according to the invention is used as the initial substance and sodium methylate is used as the reaction component, the course of the second stage of the method (a) according to the invention according to variant α can be illustrated by the following formula scheme.

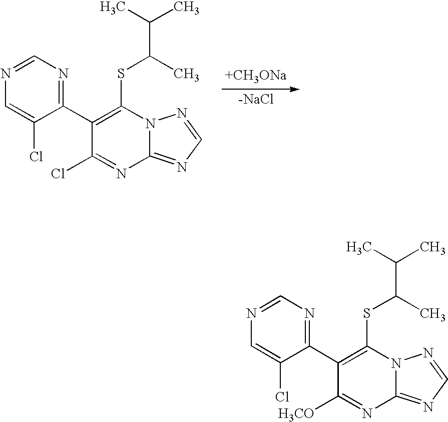

If the aforementioned compound prepared according to the first stage of the method (a) according to the invention is used as the initial substance and methyl magnesium bromide is used as the reaction component, the course of the second stage of the method (a) according to the invention according to variant β can be illustrated by the following formula scheme.

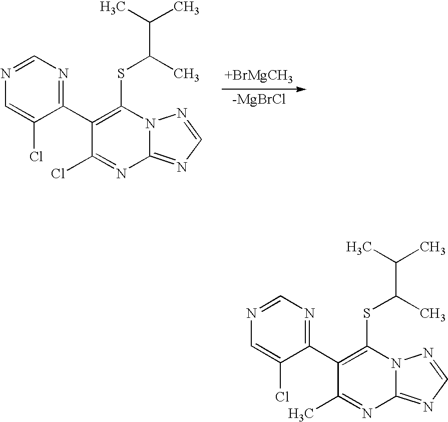

The dihalogen-triazolo-pyrimidines required as initial substances when implementing the method (a) according to the invention are generally defined by formula (II). In this formula (II) R² and R³ preferably have the same meanings as were specified previously as preferred for these residues in connection with the description of the substances of formula (I) according to the invention. X¹ preferably represents fluorine, chlorine or bromine. Y¹ preferably represents fluorine, chlorine or bromine, especially preferably fluorine or chlorine.

The dihalogen-triazolo-pyrimidines of formula (II) are new. These substances are also suitable for combating undesirable micro-organisms.

These dihalogen-triazolo-pyrimidines can be prepared by reacting

(b) Dihydroxy-triazolo-pyrimidines of the formula 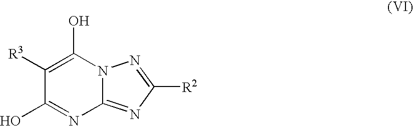
in which
R² and R³ has the meaning specified above,
with halogenating agents, optionally in the presence of a diluent.

If 6-(5-chloropyrimidin-4-yl)-[1,2,4]triazolo[1,5-a]-pyrimidin-5,7-diole is used as the initial substance and phosphorus oxychloride mixed with phosphorus pentachloride is used as the halogenating agent, the course of the method (b) according to the invention can be illustrated by the following formula scheme

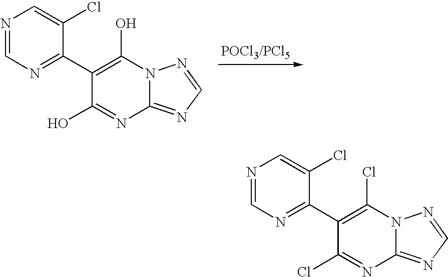

The dihydroxy-triazolopyrimidines required as initial substances when implementing the method (b) according to the invention are generally defined by formula (VI). In this formula (II) R² and R³ preferably have the same meanings as were specified previously as preferred for these residues in 5 connection with the description of the substances of formula (I) according to the invention.

The dihydroxy-triazolopyrimidines of formula (VI) are also previously unknown. They can be prepared by reacting

(c) heterocyclyl malonic esters of the formula 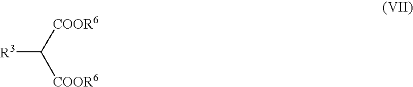
in which
R³ has the meaning specified above and
R⁶ represents alkyl with 1 to 4 carbon atoms,
with aminotriazoles of the formula 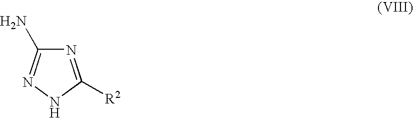
   in which
R² has the meaning specified above,
optionally in the presence of a diluent and optionally in the presence of an acid binder.

If 2-(5-chloropyrimidin-4-yl)-malonic acid dimethylester and 3-aminotriazole are used as initial substances, the course of the method (c) according to the invention can be illustrated by the following formula scheme

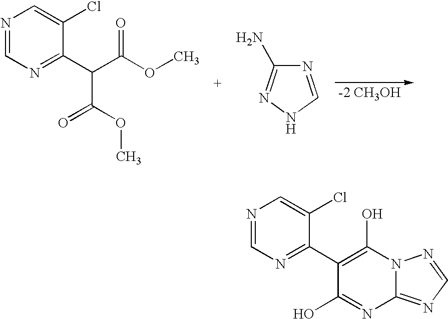

The heterocyclyl malonic esters required as initial substances when implementing the method (c) according to the invention are generally defined by formula (VII). In this formula R³ preferably has the same meanings as were specified previously as preferred for this residue in connection with the description of the substances of formula (I) according to the invention. R⁶ represents methyl or ethyl.

The heterocyclyl malonic esters of formula (VII) are partly known (cf. DE 38 20 538-A, WO 01-11 965 and WO 99-32 464).

Pyridyl malonic esters of the formula 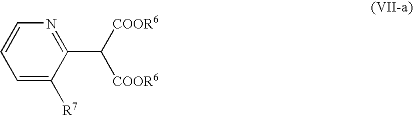
in which
R⁶ has the meaning specified above and
R⁷ represents halogen or haloalkyl.
are new.

Also new are pyrimidyl malonic esters of the formula 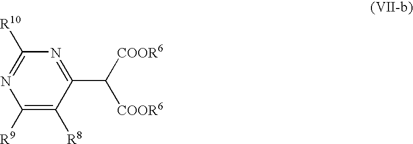
in which
R⁶ has the meaning specified above,
R⁸ represents halogen or haloalkyl and
R⁹ and R¹⁰ independently of one another represent hydrogen, fluorine, chlorine, bromine, methyl, ethyl or methoxy.

The pyridyl malonic esters of formula (VI-a) can be prepared by reacting

(d) pyridine halides of the formula 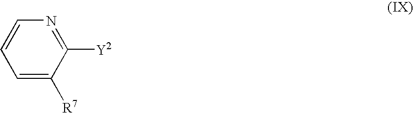
in which
R⁷ has the meaning specified above and
Y² represents halogen,
with malonic esters of the formula 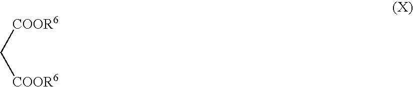
   in which
R⁶ has the meaning specified above,
optionally in the presence of a diluent, optionally in the presence of a copper salt and optionally in the presence of an acid acceptor.

If 2-chloro-3-trifluormethylpyridine and malonic acid dimethylester are used as initial substances, the course of the method (d) according to the invention can be illustrated by the following formula scheme

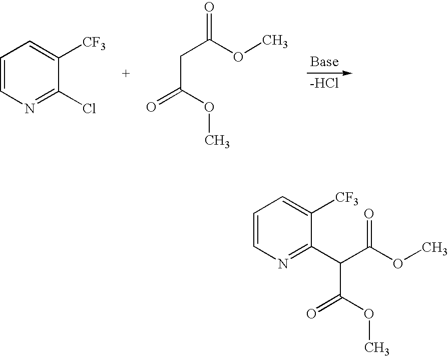

The pyridine halides required as initial substances when implementing the method (d) according to the invention are generally defined by formula (IX). In this formula R⁷ preferably represents fluorine, chlorine or trifluoromethyl. Y² preferably represents chlorine or bromine.

The pyridine halides of formula (IX) are known synthesis chemicals.

The malonic acid esters of formula (X) required as initial substances when implementing the method (d) according to the invention are also known synthesis chemicals.

The pyrimidyl malonic esters of formula (VII-b) can be prepared by reacting

(e) pyrimidine halides of the formula 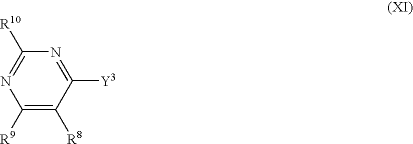
in which
R⁸, R⁹ and R¹⁰ have the meanings specified above and
Y³ represents halogen,
with malonic esters of the formula 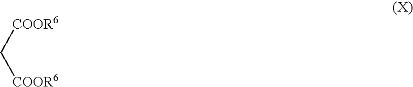
   in which
R⁶ has the meaning specified above,
optionally in the presence of a diluent, optionally in the presence of a copper salt and optionally in the presence of an acid acceptor.

If 4,5-dichloropyrimidine and malonic acid dimethylester are used as initial substances, the course of the method (e) according to the invention can be illustrated by the following formula scheme.

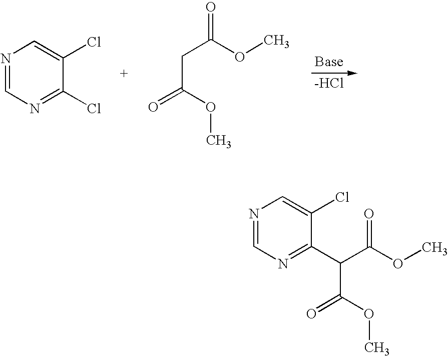

The pyrimidine halides required as initial substances when implementing the method (e) according to the invention are generally defined by formula (XI). In this formula R⁸ preferably represents fluorine, chlorine or trifluoromethyl. R⁹ and R¹⁰ preferably represent, independently of one another, hydrogen, fluorine, chlorine, bromine, methyl, ethyl or methoxy. Y³ preferably represents chlorine or bromine.

The pyrimidine halides of formula (XI) are known and can be prepared by known methods (cf J. Chem. Soc. 1955, 3478, 3481).

The aminotriazoles required as reaction components when implementing the method (c) according to the invention are generally defined by formula (VIII). In this formula R² preferably has the same meanings as were specified previously as preferred for this residue in connection with the description of the substances of formula (I) according to the invention.

The aminotriazoles of formula (VIII) are known or can be prepared by known methods (cf. DE-A 10 121 162).

All components usual for replacing hydroxy groups by halogen can be considered as halogenating agents for carrying out the method (b). Preferably used are phosphorus trichloride, phosphorus tribromide, phosphorus pentachloride, phosphorus oxychloride, thionyl chloride, thionyl bromide or mixtures thereof. The corresponding fluorine compounds of formula (II) can be prepared from the chlorine or bromine compounds by reacting with potassium fluoride.

Said halogenating agents are known.

The compounds required as reaction components when implementing the method (a) according to the invention are generally defined by formula (III). In this formula R¹ and G preferably have the same meanings as were specified previously as preferred for these residues in connection with the description of the substances of formula (I) according to the invention.

The compounds of formula (III) are known or can be prepared by known methods.

The triazolopyrimidines required as initial substances when implementing the second stage of method (a) according to the invention are generally defined by formula (Ia). In this formula R¹, R², R³ and G preferably have the same meanings as were specified previously as preferred for these residues in connection with the description of the substances of formula (I) according to the invention. X¹ preferably represents fluorine, chlorine or bromine.

The compounds required as reaction components when implementing the second stage of the method (a, variant α) according to the invention are generally defined by formula (IV). In this formula R⁴ preferably represents cyano, alkoxy with 1 to 4 carbon atoms, alkylthio with 1 to 4 carbon atoms, alkylsulphinyl with 1 to 4 carbon atoms or alkylsulphonyl with 1 to 4 carbon atoms. Me preferably represents sodium or potassium.

In formula (IV) R⁴ preferably represents cyano, methoxy or methylthio. Me especially preferably represents sodium or potassium.

The compounds of formula (IV) are known.

The Grignard compounds required as reaction components when implementing the second stage of the method (a, variant β) according to the invention are generally defined by formula (V). In this formula R⁵ preferably represents alkyl with 1 to 4 carbon atoms, especially preferably methyl. Hal both preferably and especially preferably represents chlorine or bromine.

The Grignard compounds of formula (V) are known or can be prepared by known methods, more appropriately directly before their use for further synthesis.

All usual organic solvents can be considered as diluents for carrying out the first stage of the method (a) according to the invention. Preferably used are halogenated hydrocarbons such as, for example, chlorobenzene, dichlorobenzene, dichloromethane, chloroform, tetrachloromethane, dichloroethane or trichloroethane; ethers such as diethylether, diisopropylether, methyl-t-butylether, methyl-t-amylether, dioxane, tetrahydrofuran, 1,2-dimethoxyethane, 1,2-diethoxyethane or anisol; nitriles such as acetonitrile, propionitrile, n- or i-butyronitrile or benzonitrile; amides such as N,N-dimethylformamide, N,N-dimethylacetamide, N-methylformanilide, N-methylpyrrolidone or hexamethylphosphoric acid triamide; esters such as methyl acetate or ethyl acetate; sulphoxides such as dimethylsulphoxide; sulphones such as sulpholane.

All usual inorganic or organic bases for such reactions can be considered as acid acceptors for carrying out the first stage of the method (a) according to the invention. Preferably used are alkaline-earth metal or alkali metal hydrides, hydroxides, amides, alcoholates, acetates, carbonates or hydrogen carbonates such as for example, sodium hydride, sodium amide, lithium diisopropylamide, sodium methylate, sodium ethylate, potassium tert-butylate, sodium hydroxide, potassium hydroxide, sodium acetate, potassium acetate, calcium acetate, sodium carbonate, potassium carbonate, potassium hydrogen carbonate and sodium hydrogen carbonate, and in addition ammonium compounds such as ammonium hydroxide, ammonium acetate and ammonium carbonate, as well as tertiary amines such as trimethylamine, triethylamine, tributylamine, N,N-dimethylaniline, N,N-dimethyl benzylamine, pyridine, N-methylpiperidine, N-methylmorpholine, N,N-dimethylaminopyridine, diazabicyclooctane (DABCO), diazabicyclononene (DBN) or diazabicycloundecene (DBU).

All the usual reaction accelerators for such reactions can be considered as catalysts for carrying out the first stage of the method (a) according to the invention. Preferably used are fluorides such as sodium fluoride, potassium fluoride or ammonium fluoride.

The reaction temperatures can be varied over a fairly large range for carrying out the first stage of the method (a) according to the invention. In general, temperatures between 0° C. and 150° C., preferably temperatures between 0° C. and 80° C. are used.

For carrying out the first stage of the method (a) according to the invention, generally 1 to 10 mol, preferably 1 to 3 mol of a compound of formula (III) is used per 1 mol of dihalogen-triazolo-pyrimidine of formula (II). Preparation is carried out by the usual methods.

For carrying out the second stage of the method (a, variant α) according to the invention all the usual inert organic solvents can again be considered as diluents. Preferably used are those solvents which are already specified as solvents in connection with the description of the first stage of the method (a) according to the invention.

The reaction temperatures can also be varied over a fairly large range for carrying out the second stage of the method (a, variant α) according to the invention. In general, temperatures between 0° C. and 150° C., preferably temperatures between 20° C. and 100° C. are used

For carrying out the second stage of the method (a, variant α) according to the invention triazolopyrimidine of formula (Ia) is reacted with an equivalent quantity or with an excess of a compound of formula (IV). Preparation is carried out by the usual methods.

For carrying out the second stage of the method (a, variant β) according to the invention all the usual solvents for Grignard reactions can be considered as diluents. Preferably used are ethers such as diethylether.

The reaction temperatures can be varied in a specific range for carrying out the second stage of the method (a, variant β) according to the invention. In general, temperatures between −20° C. and 80° C., preferably between 0° C. and 60° C. are used

For carrying out the second stage of the method (a, variant β) according to the invention triazolopyrimidine of formula (Ia) is reacted with an equivalent quantity or with an excess of Grignard compound of formula (V). Preparation is carried out by the usual methods.

All solvents usual for such halogenations can be considered as diluents for carrying out the method (b) according to the invention. Preferably used are halogenated aliphatic or aromatic hydrocarbons such as chlorobenzene. However, halogenating agents themselves, e.g. phosphorus oxychloride or a mixture of halogenating agents can also function as diluents.

The reaction temperatures can also be varied over a fairly large range for carrying out method (b) according to the invention. In general, temperatures between 0° C. and 150° C., preferably between 10° C. and 120° C. are used.

For carrying out method (b) according to the invention, dihydroxy-triazolpyrimidine of formula (VI) is generally reacted with an excess of halogenating agent. Preparation is carried out by the usual methods.

All the usual inert organic solvents for these reactions can be considered as diluents for carrying out method (c) according to the invention. Preferably used are alcohols such as methanol, ethanol, n-propanol, i-propanol, n-butanol and tert-butanol.

All the usual inorganic and organic bases for these reactions can be considered as acid binders for carrying out method (c) according to the invention. Preferably used are tertiary amines such as tributylamine or pyridine. Amine used in excess can also act as a diluent.

The temperatures can be varied over a fairly wide range for carrying out method (c) according to the invention. In general, temperatures between 20° C. and 200° C., preferably between 50° C. and 180° C. are used.

For carrying out method (c) according to the invention heterocyclyl malonic esters of formula (VII) and aminotriazole of formula (VIII) are generally reacted in equivalent quantities.

However, it is also possible to use one or the other component in excess. Preparation is carried out by the usual methods.

All usual inert organic solvents can be considered as diluents when implementing methods (d) and (e) according to the invention. Preferably used are halogenated hydrocarbons such as, for example, chlorobenzene, dichlorobenzene, dichloromethane, chloroform, tetrachloromethane, dichloroethane or trichloroethane; ethers such as diethylether, diisopropylether, methyl-t-butylether, methyl-t-amylether, dioxan, tetrahydrofuran, 1,2 dimethoxyethane, 1,2-diethoxyethane or anisol; nitriles such as acetonitrile, propionitrile, n- or i-butyronitrile or benzonitrile; amides such as N,N-dimethylformamide, N,N-dimethylacetanide, N-methylformanilide, N-methylpyrrolidon or hexamethylphosphoric acid triamide; sulphoxides such as dimethylsulphoxide; sulphones such as sulpholane; alcohols such as methanol, ethanol, n- or i-propanol, n-, i-, sec- or tert-butanol, ethanediol, propane-1,2-diol, ethoxyethanol, methoxyethanol, diethyleneglycolmonomethylether, diethyleneglycolmonoethylether, mixtures thereof with water or also pure water.

The usual copper salts can be considered as copper salts in each case when implementing methods (d) and (e) according to the invention. Preferably used are copper(I) chloride or copper(I) bromide.

The usual inorganic or organic bases can be considered in each case as acid acceptors when implementing methods (d) and (e) according to the invention. Preferably used are alkaline-earth metal or alkali metal hydrides, hydroxides, amides, alcoholates, acetates, carbonates or hydrogen carbonates such as, for example, sodium hydride, sodium amide, lithium diisopropylamide, sodium methylate, sodium ethylate, potassium tert-butylate, sodium hydroxide, potassium hydroxide, sodium acetate, potassium acetate, calcium acetate, sodium carbonate, potassium carbonate, potassium hydrogen carbonate and sodium hydrogen carbonate and in addition ammonium compounds such as ammonium hydroxide, ammonium acetate and ammonium carbonate as well as tertiary amines such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

The reaction temperatures can be varied over a fairly wide range when implementing methods (d) and (e) according to the invention. In general, temperatures between 0° C. and 150° C., preferably temperatures between 0° C. and 80° C. are used.

For carrying out method (d) according to the invention, generally 1 to 15 mol, preferably 1.3 to 8 mol of malonic ester of formula (X) is used per 1 mol of pyridine halide of formula (IX). Preparation is carried out by the usual methods.

For carrying out method (e) according to the invention, generally 1 to 15 mol, preferably 1.3 to 8 mol of malonic ester of formula (X) is used per 1 mol of pyrimidine halide of formula (XI). Preparation is carried out by the usual methods.

The methods according to the invention are generally carried out at atmospheric pressure. However it is also possible to work at elevated pressure.

The substances according to the invention have a strong microbicidal effect and can be used to combat undesirable micro-organisms such as fungi and bacteria, to protect plants and to protect materials.

Fungicides can be used for plant protection to combat plasmodiophoromycetes, oomycetes, chytridiomycetes, zygomycetes, ascomycetes, basidiomycetes and deuteromycetes.

Bactericides can be used in plant protection to combat Pseudomonadaceae, Rhizobiaceae, Enterobacteriaceae, Corynebacteriaceae and Streptomycetaceae.

Some pathogens of fungal and bacterial diseases which come within the above-mentioned headings may be mentioned as examples but are not restricted thereto:
Xanthomonas species such as, for example, Xanthomonas campestris pv. oryzae;
Pseudomonas species such as, for example, Pseudomonas syringae pv. lachrymans;
Erwinia species such as, for example, Erwinia amylovora;
Pythium species such as, for example, Pythium ultimum;
Phytophthora species such as, for example, Phytophthora infestans;
Pseudoperonospora species such as, for example, Pseudoperonospora humuli or Pseudoperonospora cubensis;
Plasmopara species such as, for example, Plasmopara viticola;
Bremia species such as, for example, Bremia lactucae;
Peronospora species such as, for example, Peronospora pisi or P. brassicae;
Erysiphe species such as, for example, Erysiphe graminis;
Sphaerotheca species such as, for example, Sphaerotheca fuliginea;
Podosphaera species such as, for example, Podosphaera leucotricha;
Venturia species such as, for example, Venturia inaequalis;
Pyrenophora species such as, for example, Pyrenophora teres or P. graminea (conodia form: Drechslera, Syn: Helminthosporium);
Cochliobolus species such as, for example, Cochliobolus sativus (conodia form: Drechslera, Syn: Helminthosporium);
Uromyces species such as, for example, Uromyces appendiculatus;
Puccinia species such as, for example, Puccinia recondita;
Sclerotinia species such as, for example, Sclerotinia sclerotiorum;
Tilletia species such as, for example, Tilletia caries;
Ustilago species such as, for example, Ustilago nuda or Ustilago avenae;
Pellicularia species such as, for example, Pellicularia sasakii;
Pyricularia species such as, for example, Pyricularia oryzae;
Fusarium species such as, for example, Fusarium culmorum;
Botrytis species such as, for example, Botrytis cinerea;
Septoria species such as, for example, Septoria nodorum;
Leptosphaeria species such as, for example, Leptosphaeria nodorum;
Cercospora species such as, for example, Cercospora canescens;
Alternaria species such as, for example, Alternaria brassicae;
Pseudocercosporella species such as, for example, Pseudocercosporella herpotrichoides.

The active substances according to the invention have a very good strengthening effect in plants. They are thus suited for mobilising the plant's own defences against attack by undesirable microorganisms.

Plant-strengthening (resistance-inducing) substances are to be understood in the present connection as those substances which are capable of stimulating the defence system of plants so that when inoculated subsequently with undesirable micro-organisms, the treated plants develop extensive. resistance to these micro-organisms.

Undesirable micro-organisms are to be understood in the present case as phytopathogenic fungi, bacteria and viruses. The substances according to the invention can thus be used to protect plants from attack by said pathogens over a certain time following treatment. The period of time within which protection is afforded generally extends from 1 to 10 days, preferably 1 to 7 days after treatment of the plants with the active substances.

The good phytotolerance of the active substances in the concentrations necessary to combat plant diseases means that above-ground parts of plants, plants and seeds and the soil can be treated. In this situation, the active substances according to the invention can be used particularly successfully to combat cereal diseases such as Erysiphe species, diseases in wine-growing, fruit and vegetable growing such as Botrytis, Venturia, Sphaerotheca and Podosphaera species.

The active substances according to the invention are suitable for increasing the crop yield. They are also less toxic and exhibit good phytotolerance.

The active substances according to the invention can optionally be used in certain concentrations and quantities as herbicides, to influence plant growth and also to combat animal pests. They can also optionally be used as intermediate and initial products for synthesising further active substances.

All plants and parts of plants can be treated according to the invention. In this context, plants are understood as all plants and plant populations such as desired and undesired wild plants and cultivated plants (including naturally occurring cultivated plants). Cultivated plants can be plants which can be obtained by conventional cultivation and optimisation methods or by biotechnology and gene technology methods or combinations of these methods, including transgenic plants and including plant varieties which can or cannot be protected by variety property rights. Parts of plants should be understood as above-ground and underground parts and organs of plants such as scion, leaf, flower and root, with leaves, needles, stakes, stems, flowers, fruiting bodies, fruits and seeds as well as roots, bulbs and rhizomes being cited as examples. Parts of plants also include harvested material as well as vegetative and generative replicative material, for example, cuttings, bulbs, rhizomes, scions and seeds.

The treatment according to the invention of plants and parts of plants with the active substances is carried out directly or by acting on their surroundings, habitat or storage area using the conventional treatment methods, e.g. by dipping, spraying, vaporising, nebulising, scattering, sprinkling and in the case of replicative material, especially in seeds, furthermore by single- or multilayer coating.

For the protection of materials, the substances according to the invention can be used to protect technical materials against attack and destruction by undesirable micro-organisms.

Technical materials are to be understood in the present connection as non-living materials which have been prepared for use in technology. For example, technical materials which are to be protected from microbial modification or destruction by active substances according to the invention are adhesives, glues, paper and cardboard, textiles, leather, wood, coating agents and plastic articles, cooling lubricants and other materials which can be attacked or destroyed by microorganisms. Within the scope of materials to be protected, mention may also be made of parts of production installations, for example, cooling water circuits which can be adversely affected by multiplication of micro-organisms. Within the scope of the present invention, technical materials are preferably adhesives, glues, paper and cardboard, leather, wood, coating agents, cooling lubricants and heat-transfer liquids, especially preferably wood.

As micro-organisms which can bring about a degradation or modification of technical materials, mention may be made, for example, of bacteria, fungi, yeasts, algae and slime organisms. The active substances according to the invention preferably act against fungi, especially moulds, wood-staining and wood-destroying fungi (basidiomycetes) and against slime organisms and algae.

Micro-organisms of the following genus may be mentioned as examples:
Alternaria such as Alternaria tennis,
Aspergillus such as Aspergillus niger,
Chaetomium such as Chaetomium globosum,
Coniophora such as Coniophora puetana,
Lentinus such as Lentinus tigrinus,
Penicillium such as Penicillium glaucum,
Polyporus such as Polyporus versicolor,
Aureobasidium such as Aureobasidium pullulans,
Sclerophoma such as Sclerophoma pityophila,
Trichorma such as Trichorma viride,
Escherichia such as Escherichia coli,
Pseudomonas such as Pseudomonas aeruginosa,
Staphylococcus such as Staphylococcus aureus.

Depending on their respective physical and/or chemical properties, the active substances can be converted into the usual formulations such as solutions, emulsions, suspensions, powders, foams, pastes, granules, aerosols, ultrafine encapsulations in polymer materials, and in coating compounds for seeds, as well as ULV cold and warm mist formulations.

These formulations are produced in a known fashion, e.g. by mixing the active substances with extenders, i.e. liquid solvents, pressurised liquefied gases and/or solid carriers, optionally using surfactants, i.e. emulsifiers and/or dispersants and/or foaming agents. If water is used as an extending agent, for example, organic solvents can also be used as auxiliary solvents. Possible liquid solvents are substantially: aromatic compounds such as xylene, toluene or alkylnaphthalene, chlorinated aromatic compounds or chlorinated aliphatic hydrocarbons such as chlorobenzenes, chloroethylenes or methylene chloride, aliphatic hydrocarbons such as cyclohexane or paraffins, e.g. petroleum fractions, alcohols such as butanol or glycol as well as their ethers and esters, ketones such as acetone, methylethylketone, methylisobutylketone or cyclohexanone, strongly polar solvents such as dimethylformamide and dimethylsulphoxide as well as water. Liquefied gaseous extenders or carriers means those liquids which are gaseous at normal temperature and at normal pressure e.g. aerosol propellant gases such as halogen hydrocarbons as well as butane, propane, nitrogen and carbon dioxide. Possible solid carriers are: e.g. natural ground stone such as kaolin, clay, talc, chalk, quartz, attapulgite, montmorillonite or diatomataceous earth and synthetic ground stone such as highly disperse silicic acid, aluminium oxide and silicates. Possible solid carriers for granules are: e.g. broken and fractionated natural stone such as calcite, pumice, sepiolith, dolomite as well as synthetic granules of inorganic or organic flours as well as granules of organic material such as sawdust, coconut shells, maize cobs and tobacco stalks. Possible emulsifiers and/or foaming agents are: e.g. non-ionogenic and anionic emulsifiers such as polyoxyethylene fatty acid esters, polyoxyethylene fatty alcohol ethers e.g. alkylarylpolyglycolether, alkylsulphonates, alkylsulphates, arylsulphonates and protein hydrolysates. Possible dispersants are: e.g: lignin sulphite waste and methylcellulose.

Adhesives such as carboxymethylcellulose, natural and synthetic powder, granular or latex-form polymers such as gum arabic, polyvinylalcohol, polyvinylacetate, as well as natural phospholipids such as cephaline and lecithin, and synthetic phospholipids can be used in the formulations. Further additives can be mineral and vegetable oils

Dyes such as inorganic pigments, e.g. iron oxide, titanium oxide, ferrocyan blue and organic dyes such as alizarine, azo and metal phthalocyanine dyes and trace nutrients such as salts of iron, manganese, boron, copper, cobalt, molybdenum and zinc can be used.

The formulations generally contain between 0.1 and 95 weight percent of active substance, preferably between 0.5 and 90%.

The active substances according to the invention can be used as such or in their formulations also mixed with known fungicides, bactericides, acaricides, nematicides or insecticides in order to thus broaden the spectrum of action, for example, or avoid the development of resistance. In many cases, synergistic effects are achieved in this situation, i.e., the efficiency of the mixture is higher than the efficiency of the individual components.

The following compounds can be considered as mixing partners, for example:

Fungicides:
2-phenylphenol; 8-hydroxychinolinsulphate;
acibenzolar-S-methyl; aldimorph; amidoflumet; ampropylfos; ampropylfos potassium; andoprim; anilazine; azaconazole; azoxystrobin;
benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine;
calcium-polysulphide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram;
Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon;
edifenphos; epoxiconazole; ethaboxam; ethirimol; etridiazole;
famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulphamide; flutolanil; flutriafol; folpet; fosetyl-Al; fosetyl-sodium; fuberidazole; furalaxyl; furametpyr; furcarbanil; furmecyclox;
guazatine;
hexachlorobenzene; hexaconazole; hymexazol;
imazalil; imibenconazole; iminoctadine triacetate; iminoctadine tris(albesil; iodocarb; ipconazole; iprobenfos; iprodione; iprovalicarb; irumamycin; isoprothiolane; isovaledione;
kasugamycin; kresoxim-methyl;
mancozeb; maneb; meferimzone; mepanipyrim; mepronil; metalaxyl; metalaxyl-M; metconazole; methasulphocarb; methfuroxam; metiram; metominostrobin; metsulphovax; mildiomycin; myclobutanil; myclozolin;
natamycin; nicobifen; nitrothal-isopropyl; noviflumuron; nuarimol;
ofurace; orysastrobin; oxadixyl; oxolinic acid; oxpoconazole; oycarboxin; oxyfenthiin;
paclobutrazol; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrine;
quinconazole; quinoxyfen; quintozene;
simeconazole; spiroxamine; sulphur;
tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole;
uniconazole;
validamycin A; vinclozolin;
zineb; ziram; zoxamide;
(2S)-N-[2-[4-[[3-(4-chlorophenyl)-2-propinyl]oxy]-3-methoxyphenyl]ethyl]-3-methyl-2-[(methylsulphonyl)amino]-butanamide;
1-(1-naphthalenyl)-1H-pyrrol-2,5-dione;
2,3,5,6-tetrachloro-4-(methylsulphonyl)-pyridine;
2-amino-4-methyl-N-phenyl-5-thiazolcarboxamide;
2-chloro-N-(2,3-dihydro-1,1,3-trimethyl-1H-inden4-yl)-3-pyridine carboxamide;
3,4,5-trichloro-2,6-pyridine dicarbonitrile;
actinovates;
cis-1-(4-chlorophenyl)-2-(1H-1,2,4-triazol-1-yl)-cycloheptanol;
methyl 1-(2,3-dihydro-2,2-dimethyl-1H-inden-1-yl)-1H-imidazol-5-carboxylate;
monopotassium carbonate;
N-(6-methoxy-3-pyridinyl)-cyclopropane carboxamide;
sodium tetrathiocarbonate;
as well as copper salts and preparations such as Bordeaux mixture; copper hydroxide; copper naphthenate; copper oxychloride; copper sulphate; cufraneb; copper oxide; mancopper; oxine-copper.

Bactericides:

Bronopol, dichlorophen, nitrapyrin, nickel-dimethyldithiocarbamate, kasugamycin, octhilinon, furancarbonic acid, oxytetracyclin, probenazol, streptomycin, tecloftalam, copper sulphate and other copper preparations.

Insecticides/Acaricides/Nematicides:

Abamectin, ABG-9008, acephate, acequinocyl, acetamiprid, acetoprole, acrinathrin, AKD-1022, AKD-3059, AKD-3088, alanycarb, aldicarb, aldoxycarb, allethrin, allethrin 1R-isomers, alphacypermethrin (alphamethrin), amidoflumet, aminocarb, amitraz, avermectin, AZ-60541, azadirachtin, azamethiphos, azinphos-methyl, azinphos-ethyl, azocyclotin,
Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, Baculoviruses, Beauveria bassiana, Beauveria tenella, bendiocarb, benfuracarb, bensultap, benzoximate, beta-cyfluthrin, beta-cypermethrin, bifenazate, bifenthrin, binapacryl, bioallethrin, bioallethrin-S-cyclopentyl-isomer, bioethanomethrin, biopermethrin, bioresmethrin, bistrifluron, bpmc, brofenprox, bromophos-ethyl, bromopropylate, bromfenvinfos (-methyl), BTG-504, BTG-505, bufencarb, buprofezin, butathiofos, butocarboxim, butoxycarboxim, butylpyridaben,
cadusafos, camphechlor, carbaryl, carbofuran, carbophenothion, carbosulphan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifos-methyl, chlorpyrifos (-ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, cydia pomonella, cyfluthrin, cyhalothrin, cyhexatin, cypermethrin, cyphenothrin (1R-trans-isomer), cyromazine,
DDT, deltamethrin, demeton-s-methyl, demeton-s-methylsulphon, diafenthiuron, dialifos, diazi-non, dichlofenthion, dichlorvos, dicofol, dicrotophos, dicyclanil, diflubenzuron, dimethoate, di-methylvinphos, dinobuton, dinocap, dinotefuran, diofenolan, disulphoton, docusat-sodium, dofenapyn, DOWCO-439,
eflusilanate, emamectin, emamectin-benzoate, empenthrin (1R-isomer), endosulphan, entomopthora spp., EPN, esfenvalerate, ethiofencarb, ethiprole, ethion, ethoprophos, etofenprox, etoxazole, etrimfos,
famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulphothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb,
gamma-HCH, gossyplure, grandlure, granulose viruses,
halfenprox, halofenozide, HCH, HCN-801, heptenophos, hexaflumuron, hexythiazox, hydramethylnone, hydroprene,
IKA-2002, imidacloprid, imiprothrin, indoxacarb, iodofenphos, iprobenfos, isazofos, isofenphos, isoprocarb, isoxathion, ivermectin,
japonilure,
kadethrin, nuclear polyhedrosis viruses, kinoprene,
lambda-cyhalothrin, lindane, lufenuron,
malathion, mecarbam, mesulphenfos, metaldehyd, metam-sodium, methacrifos, methamidophos, metharhizium anisopliae, metharhizium flavoviride, methidathion, methiocarb, methomyl, methoprene, methoxychlor, methoxyfenozide, metolcarb, metoxadiazone, mevinphos, milbemectin, milbemycin, MKI-245, MON-45700, monocrotophos, moxidectin, MTI-800,
naled, NC-104, NC-170, NC-184, NC-194, NC-196, niclosamide, nicotine, nitenpyram, nithiazine, NNI-0001, NNI-0101, NNI-0250, NNI-9768, novaluron, noviflumuron,
OK-5101, OK-5201, OK-9601, OK-9602, OK-9701, OK-9802, omethoate, oxamyl, oxydemetonmethyl,
Paecilomyces fumosoroseus, parathion-methyl, parathion (-ethyl), permethrin (cis-, trans-), petroleum, PH-6045, phenothrin (1R-trans isomer), phenthoate, phorate, phosalone, phosmet, phosphamidon, phosphocarb, phoxim, piperonyl butoxide, pirimicarb, pirimiphos-methyl, pirimiphosethyl, prallethrin, profenofos, promecarb, propaphos, propargite, propetamphos, propoxur, prothiofos, prothoate, protrifenbute, pymetrozine, pyraclofos, pyresmethrin, pyrethrum, pyridaben, pyridalyl, pyridaphenthion, pyridathion, pyrimidifen, pyriproxyfen,
quinalphos,
resmethrin, RH-5849, ribavirin, RU-12457, RU-15525,
S-421, S-1833, salithion, sebufos, SI-0009, silafluofen, spinosad, spirodiclofen, spiromesifen, sulphluramid, sulphotep, sulprofos, SZI-121,
tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (1R-isomer), tetrasul, theta-cypermethrin, thiacloprid, thiamethoxam, thiapronil, thiatriphos, thiocyclam hydrogen oxalate, thiodicarb, thiofanox, thiometon, thiosultap-sodium, thuringiensin, tolfenpyrad, tralocythrin, tralomethrin, transfluthrin, triarathene, triazamate, triazophos, triazuron, trichlophenidine, trichlorfon, triflumuron, trimethacarb,
vamidothion, vaniliprole, verbutin, Verticillium lecanii,
WL-108477, WL-40027,
YI-5201, YI-5301, YI-5302,
XMC, xylylcarb,
ZA-3274, Zeta-Cypermethrin, Zolaprofos, ZXI-8901,
the compound 3-methyl-phenyl-propylcarbamate (Tsumacide Z),
the compound 3-(5-chloro-3-pyridinyl)-8-2,2,2-trifluorethyl)-8-azabicyclo[3.2.1octan-3-carbonitrile (CAS Reg. No. 185982-80-3) and the corresponding 3-endo-isomers (CAS Reg. No. 185984-60-5) (cf. WO-96/37494, WO-98/25923),
as well as preparations containing plant extracts having an insecticidal effect, nematodes, fungi or viruses.

A mixture with other known active substances such as herbicides or with fertilisers and growth regulators, safeners or semiochemicals is also possible.

The compounds of formula (I) according to the invention also exhibit very good antimycotic effects. They have a very broad antimycotic spectrum of action, especially against dermatophytes and yeast fungi, mould and diphasic fungi (e.g. towards Candida species such as Candida albicans, Candida glabrata) as well as Epidermophyton floccosum, Aspergillus species such as Aspergillus niger and Aspergillus fumigatus, Trichophyton species such as Trichophyton mentagrophytes, Microsporon species such as Microsporon canis and audouinii. The listing of these fungi in no way represents any restriction of the mycotic spectrum which can be covered but is merely of an explanatory nature.

In addition, the compounds of formula (I) according to the invention are also suitable for suppressing the growth of tumour cells in humans and mammals. This is based on an interaction of the compounds according to the invention with tubulin and microtubuli and by promoting microtubuli polymerisation.

An effective quantity of one or more compounds of formula (I) or pharmaceutically tolerable salts thereof can be administered for this purpose.

The active substances can be used as such, in the form of their formulations or forms of application derived therefrom such as ready-to-use solutions, suspensions, spray powder, pastes, soluble powders, dusting agents and granules. Application takes place in the usual manner e.g. by pouring, splashing, spraying, scattering, dusting, foaming, brushing etc. It is further possible to apply the active substances by the ultra-low volume method or to inject the active substance preparation or the active substance itself into the soil. The seeds of plants can also be treated.

When the active substances according to the invention are used as fungicides, the quantities used can be varied over a fairly large range according to the type of application. When parts of plants are treated, the quantities of active substance used are generally between 0.1 and 10,000 g/ha, preferably between 10 and 1000 g/ha. When seeds are treated, the quantities of active substance used are generally between 0.001 and 50 g per kilogram of seed, preferably between 0.01 and 10 g per kilogram of seed. When the soil is treated, the quantities of active substance used are generally between 0.1 and 10,000 g/ha, preferably between 10 and 5000 g/ha.

As has already been mentioned above, all plants and parts thereof can be treated according to the invention. In a preferred embodiment plant species and plant varieties and parts thereof which occur in the wild or are obtained by conventional biological cultivation methods such as crossing or protoplast fusion are treated. In a further preferred embodiment transgenic plants and plant varieties and parts thereof obtained by gene technology methods (genetically modified organisms) optionally in conjunction with conventional methods are treated. The term “parts” or “parts of plants” or “plant parts” has been explained above.

It is especially preferable if plants of the respectively commercially available plant varieties or those in common use are treated according to the invention. Plant varieties is understood as plants with new properties (“traits”) which have been cultivated by conventional cultivation, by mutagenesis or by recombinant DNA techniques. These can be varieties, strains, biotypes or genotypes.

According to the plant species or plant varieties, their location and growth conditions (soils, climate, vegetation period, nourishment), superadditive (“synergistic”) effects can occur as a result of the treatment according to the invention. Thus, for example, it is possible to achieve reduced quantities used and/or extensions of the spectrum of action and/or an intensification of the action of the substances and means used according to the invention, better plant growth, increased tolerance to high or low temperatures, increased tolerance to drought or towards water and soil salt content, increased flowering performance, easier harvesting, faster ripening, higher crop yields, higher quality and/or higher nutritional value of the harvest products, higher storage capacity and/or processability of the harvest products which go beyond the effects which are actually expected.

Preferred transgenic (obtained by gene technology) plants or plant varieties to be treated according to the invention include all plants obtained by gene technology modification of genetic material which imparts to these plants particularly advantageous valuable properties (“traits”). Examples of such properties are better plant growth, increased tolerance to high or low temperatures, increased tolerance to drought or towards water and soil salt content, increased flowering performance, easier harvesting, faster ripening, higher crop yields, higher quality and/or higher nutritional value of the harvest products, higher storage capacity and/or processability of the harvest products. Further and especially emphasised examples of such properties are an increased defence of the plants towards animal and microbial pests, and towards insects, mites, phytopathogenic fungi, bacteria and/or viruses and an increased tolerance of the plants towards specific herbicidal active substances. As examples of transgenic plants, mention is made of important cultivated plants such as cereals (wheat, rice), maize, soya, potato, cotton, tobacco, rape and fruit plants (with the fruits apples, pears, citrus fruits and grapes), with maize, soya, potato, cotton, tobacco and rape being particularly emphasised. Properties (“traits”) particularly emphasised include the increased defence of the plants toward insects, arachnids, nematodes and snails as a result of toxins produced in the plants, especially those produced in the plants (hereinafter called “Bt plants”) by the genetic material from Bacillus Thuringiensis (e.g. by the genes CryIA(a), CryIA(b), CryIA(c), CryIIA, CryIIIA, CryIIIB2, Cry9c Cry2Ab, Cry3Bb and CryIF and combinations thereof). Properties (“traits”) especially emphasised include the increased defence of plants towards fungi, bacteria, viruses through systemically acquired resistance (SAR), systemin, phytoalexins, elicitors as well as resistance genes and suitably expressed proteins and toxins. Properties (“traits”) especially emphasised include the increased tolerance of the plants toward certain herbicidal active substances, for example, imidazolinones, sulphonyl ureas, glyphosate or phosphinotricin (e.g. “PAT”-Gene). The genes imparting the desired properties (“traits”) in each case can also occur in combination with one another in transgenic plants. As examples of “Bt plants” mention may be made of maize varieties, cotton varieties, soya varieties and potato varieties which are sold under the tradenames YIELD GARD® (e.g. maize, cotton, soya), KnockOut® (e.g. maize), StarLink® (e.g. maize), Bollgard® (cotton), Nucoton® (cotton) and NewLeaf® (potato). As examples of herbicide-tolerant plants mention may be made of maize varieties, cotton varieties and soya varieties which are sold under the tradenames Roundup Ready® (tolerance towards glyphosates e.g. maize, cotton, soya), Liberty Link® (tolerance towards phosphinotricin, e.g. rape), IMI® (tolerance towards imidazolinones) and STS® (tolerance towards sulphonyl ureas, e.g. maize). As herbicide-resistant (cultivated conventionally for herbicide tolerance) plants, mention may also be made of varieties (e.g. maize) sold under the name Clearfield®. Naturally these statements also apply to varieties of plants developed in the future or coming onto the market in the future having these genetic properties (“traits”) or those developed in the future.

The plants listed can especially advantageously be treated with compounds of the general formula (I) or active-substance mixtures according to the invention. The preferred ranges given above for the active substances or mixture also apply to the treatment of these plants. The treatment of plants with the compounds or mixtures specially mentioned in the present text is particularly emphasised.

The preparation and use of the active substances according to the invention is deduced from the following examples.

### PREPARATION EXAMPLES

### Example 1

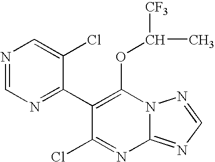

(Method a)

0.1 g of potassium fluoride is added to a solution of 0.3 g (0.995 mmol) of 5,7-dichloro-6-(5-chloro-pyrimidin-4-yl)[1,2,4]-triazolo[1,5-a]pyrimidine in 10 ml acetonitrile, stirred for 2 hours at 80° C. and then cooled to room temperature. 0.22 g (1.9 mmol) of 2,2,2-trifluoro-isopropanol and 0.3 g (2.2 mmol) of potassium carbonate is added to the solution and stirred for 4 hours at 80° C. The reaction mixture is then concentrated at reduced pressure. The remaining residue is mixed with 30 ml of water and 30 ml of dichloromethane. The organic phase is separated, dried over sodium sulphate and then concentrated at reduced pressure. The remaining residue is filtered with cyclohexane/ethylacetate (5:1 to 1:1) over a short column using silica gel. 0.05 g (11.9% of theoretical yield) of 5-chloro-6-(5-chloro-pyrimidin4-yl)-7-(2,2,2-trifluoro-isopropoxy)[1,2,4]triazolo[1,5-a]pyrimidine is obtained.

HPLC: logP=2.43

### Example 2

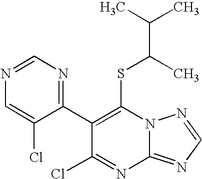

Method (a)

0.12 g of potassium fluoride is added to a solution of 0.3 g (0.995 mmol) of 5,7-dichlor-6-(5-chloro-4-pyrimidinyl)[1,2,4]triazolo[1,5-a]pyrimidine in 10 ml acetonitrile, stirred for 2 hours a 80° C. and then cooled to room temperature. 0.114 g (1.09 mmol) of 3-methyl-2-butanethiol and 0.2 g of potassium carbonate is added to the solution and stirred for 3 hours at room temperature. The reaction mixture is then concentrated at reduced pressure. The remaining residue is mixed with 30 ml of water and 30 ml of dichloroethane. The organic phase is separated, dried over sodium sulphate and then concentrated at reduced pressure. 0.17 g (42% of theoretical yield) of 5-chloro-6-5-chloro-4-pyrimidinyl)-7-[(1,2-dimethylpropyl)sulphanyl][1,2,4]triazolo[1,5-a]pyrimidine is obtained.

HPLC: logP=3.07

The compounds of formula (I) given in the following table 1 are also obtained by the methods specified previously.

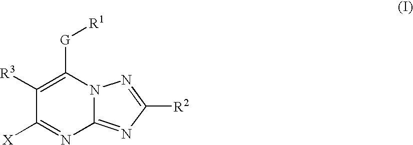

TABLE 1

| Exam- | | | | | | |
|---|---|---|---|---|---|---|
| ple | | | | | | |
| No. | R¹ | R² | R³ | G | X | logP |
| 3 | 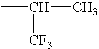 | 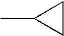 | 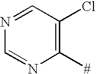 | O | Cl | 3.21 |
| 4 | 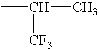 | —CH(CH₃)₂ | 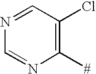 | O | Cl | 3.49 |

# indicates the linking point
*) The LogP values were determined in accordance with EEC-Directive 79/831 Annex V. A8 by HPLC (Gradient method, acetonitrile/0.1% aqueous phosphoric acid)

### Preparation of Initial Products of Formula (II)

### Example 5

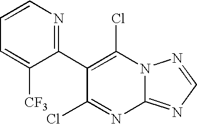

Method (b)

8 g (16 mmol) of 6-(3-trifluoromethyl-pyridin-2-yl)-[1,2,4]triazolo[1,5-a]pyrimidin-5,7-diole is mixed with 12 ml of phosphorus oxychloride. To this is added 2.7 g of phosphorus pentachloride in portions. The mixture is heated for 2 hours under reflux. After cooling to room temperature, the reaction mixture is concentrated at reduced pressure. The remaining residue is mixed with 100 ml of water and extracted three times with 100 ml of dichloromethane in each case. The combined organic phases are washed twice with 50 ml of water, dried over sodium sulphate and concentrated at reduced pressure. The residue is chromatographed with dichloromethane/methyl-t-butyl ether (95:5) on silica gel. 1.4 g (25.7% of theoretical yield) of 5,7-dichloro-6-(3-trifluoromethyl-pyridin-2-yl)-[1,2,4]triazolo[1,5-a]pyrimidine is obtained.

HPLC: logP=1.97

### Example 6

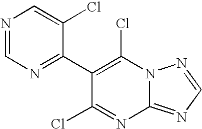

Method (b)

8 g (16 mmol) of 6-(5-chloro-4-pyrimidinyl)[1,2,4]triazolo[1,5-a]pyrimidin-5,7-diole is mixed with 25 ml of phosphorus oxychloride. 3.1 g of phosphorus pentachloride is added in portions. The mixture is stirred for 3 hours at 110° C. After cooling to room temperature, the reaction mixture is mixed with 300 ml of water and extracted three times with 100 ml of dichloromethane in each case. The combined organic phases are dried over sodium sulphate and concentrated at reduced pressure. The residue is chromatographed with hexane/ethyl acetate (9:1-5:1) on silica gel. 1.4 g (25.7% of theoretical yield) of 5,7-dichloro-6-(5-chloro-4-pyrimidinyl)[1,2,4]triazolo[1,5-a]pyrimidine is obtained.

HPLC: logP=1.43

### Example 7

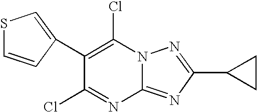

A mixture of 2.0 g (10.74 mmol) of 2-thienyl-malonic acid and 1.33 g (10.74 mmol) of 3-amino-5-cyclopropyl-1,2,4-triazol is mixed with 41.13 g (286 mmol) of phosphorus oxychloride at room temperature whilst agitating for 2 minutes. It is then heated to 90° C. for 18 hours and cooled to room temperature. The reaction mixture is added to 250 ml of iced water and the suspension thus obtained is stirred for 1 hour. Extraction is then carried out and washing with 50 ml of water. For further purification the product is suspended in 50 ml of cyclohexane/ethyl acetate=1:1 and briefly boiled, then cooled down, extracted over a short silica gel column and washed 8 times with 50 ml of cyclohexane/ethyl acetate=1:1 in each case. The filtrate is dried over sodium sulphate and then filtered again. The filter residue is re-washed using a little cyclohexane/ethyl acetate=1:1. The complete filtrate is concentrated at reduced pressure. 1.73 g (50.7% of theoretical yield) of 5,7-dichloro-2-cyclopropyl-6-(thien-3-yl)-[1,2,4]triazolo[1,5-a]pyrimidine is obtained in the form of a beige-coloured solid.

### Example 8

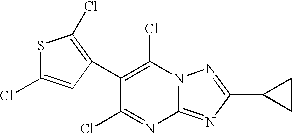

A stream of chlorine gas is fed into a solution of 6.0 g (19.28 mmol) of 5,7-dichloro-2-cyclopropyl-6-(thien-3-yl)-[1,2,4]triazolo[1,5-a]pyrimidine in 80 ml of acetic acid for 2 hours at room temperature. The reaction mixture is then concentrated at reduced pressure. The remaining residue is chromatographed with cyclohexane/ethyl acetate=2:1 on silica gel. The residue obtained after concentrating the eluate is mixed with cyclohexane/ethyl acetate=1:1, then extracted and dried. After concentrating at reduced pressure, the previously obtained mother liquor is chromatographed again with cyclohexane/ethyl acetate=1:1 on silica gel. In this way, 2.7 g (50.5% of theoretical yield) of 5,7-dichloro-2-cyclopropyl-6-(2,5-dichloro-thien-3-yl)-[1,3,4]triazolo[1,5-a]pyrimidine is obtained.

### Example 9

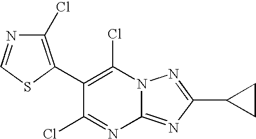

A solution of 17.0 g (54.89 mmol) of 2-cyclopropyl-6-(4-chloro-thiazol-5-yl)-[1,2,4]triazolo[1,5-a]pyrimidin-5,7-diol in 51.2 ml of phosphorus oxychloride is mixed in portions With 5.72 g (27.44 mmol) of phosphorus pentachloride whilst agitating at room temperature. After addition has been completed, the reaction mixture is stirred for 3 hours at 110° C., then cooled to room temperature and added to iced water. Extraction is then performed several times using dichloromethane, the organic phase is dried over sodium sulphate and concentrated at reduced pressure. The remaining residue is chromatographed with cyclohexane/ethyl acetate=3:1 on silica gel. In this way 0.35 g (1.66% of theoretical yield) of 5,7-dichloro-2-cyclopropyl-6-(4-chloro-thiazol-5-yl)-[1,2,4]-triazolo[1,5-a]pyrimidine is obtained.

HPLC: logP=2.46

### Preparation of Initial Products of Formula (VI[)

### Example 10

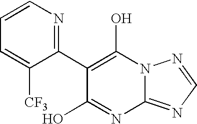

Method (c)

5.5 g (19.84 mmol) of 2-(3-trifluoromethyl-pyridin-2-yl)-malonic acid dimethylester and 1.67 g (19.84 mmol) of 3-amino-1,2,4-triazole are stirred in 5.2 ml of tributylamine for 2 hours at 180° C. The methanol produced during the reaction is continuously distilled off. After cooling, the desired product is separated from the tributylamine. The tributylamine is decanted off and the product 6-(3-trifluoromethyl-pyridin-2-yl)-[1,2,4]triazolo[1,5-a]pyrimidin-5,7-diole (yield: about 8 g, 60% purity) is used without further purification in the next reaction step.

HPLC: log=−0.23

### Example 11

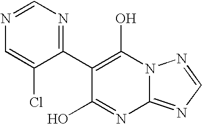

Method (c)

10 g (40.9 mmol) of 2-5-chloro-pyrimidin-4-yl)-malonic acid dimethylester and 3.44 g (40.9 mmol) of 3-amino-1,2,4-triazol are stirred in 10.7 ml of tributylamine for 2 hours at 185° C. The methanol produced during the reaction is continuously distilled off. After cooling, the desired product is separated from the tributylamine. The tributylamine is decanted off and the product 6-(5-chloro-4-pyrimidinyl)[1,2,4]triazolo[1,5-a]pyrimidin-5,7-diole (yield: about 15 g, 11% purity, about 15% of theoretical yield) is used without further purification in the next reaction step.

HPLC: logP=−0.23

### Example 12

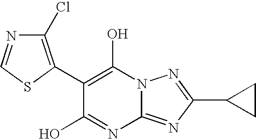

A mixture of 8.5 g (34.05 mmol) of 2-(4-chloro-thiazol-5-yl)-malonic acid dimethylester, 4.23 g (34.05 mmol) of 3-amino-5-cyclopropyl-1,2,4-triazol and 8.92 ml of tri-n-butylamine is stirred for 2 hours at 185° C. The methanol produced during the reaction is continuously distilled off. After cooling, the separated tri-n-butylamine is decanted off. In this way 18 g of a product which comprises 64% 2-cyclopropyl-6-(4-chloro-thiazol-5-yl)-[1,2,4]triazolo[1,5-a]pyrimidin-5,7-diol according to HPLC is obtained.

HPLC: logP=0.10

### Preparation of Initial Products of Formula (VII-a)

### Example 13

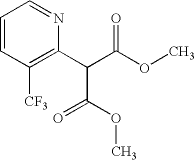

Method (d)

9 g (207 mmol) of 60% sodium hydride suspension is suspended in 300 ml of dioxane. To this 27.29 g (206.6 mmol) of malonic acid dimethylester is added dropwise at 55-60° C. and stirred for a further 30 minutes at the same temperature. After adding 8.18 g (82.63 mmol) of copper(I) chloride, the mixture is heated to 80° C. and then 15 g (82.63 mmol) of 2-chloro-3-trifluoromethylpyridine is added dropwise. The reaction mixture is now stirred for another 14 hours at 100° C. After subsequent cooling to 15-20° C., concentrated hydrochloric acid is slowly added dropwise until the mixture shows an acidic reaction. Now 600 ml of water and 300 ml of dichloromethane are added and insoluble components are filtered off. The organic phase is separated from the filtrate, dried over sodium sulphate and concentrated at reduced pressure. The residue is chromatographed with hexane/ethyl acetate (4:1) on silica gel. 10.1 g (40% of theoretical yield) of 2-[(3-trifluoromethyl)-pyridin-2-yl]-malonic acid dimethylester is obtained.

HPLC: logP=2.05

### Preparation of Initial Products of Formula (VII-b)

### Example 14

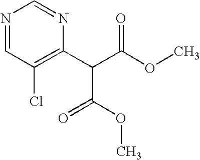

Method (e)

2.6 g (65.4 mmol) of 60% sodium hydride suspension is suspended in 100 ml of tetrahydrofuran. To this is added 6.9 g (52.4 mmol) of malonic acid dimethylester at 0C ° C. and stirred for 0.5 hours at the same temperature. A solution of 6.5 g (43.63 mmol) of 4,5-dichloropyrimidine is then added dropwise into 50 ml of tetrahydrofuran and stirred for a further 3 hours at room temperature. 150 ml of 1N hydrochloric acid is then added dropwise and then extracted with 100 ml of dichloro-methane. The organic phase is separated, dried over sodium sulphate and concentrated at reduced pressure. The residue is chromatographed with methyl-t-butyl ether/petroleum ether (1:9) on silica gel. 7 g (65.6% of theoretical yield) of 2-(5-chloro-pyrimidin-4-yl)-malonic acid dimethylester is obtained.

HPLC: logP=1.33

### Preparation of 4,5dichloropyrimidine

### Example 15

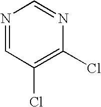

1.6 ml of dimethylamine is added to a solution of 112.5 g (673.7 mmol) of 5-chloro-6-oxo-1,6-dihydropyrimidin-1-ium chloride in 630 ml of phosphorus oxychloride and heated for 3 hours under reflux. The excess phosphorus oxychloride is then distilled off at reduced pressure. After cooling, the residue is poured onto 1.5 l of iced water and extracted with 500 ml of dichloromethane, the organic phase is dried over sodium sulphate and concentrated at reduced pressure. 72.3 g (66.3% of theoretical yield) of 4,5-dichloropyrimidine is obtained.

HPLC: logP=1.35

### Preparation of 5chloro-6-oxo-1.6-dihydropyrimidin-1-ium chloride

### Example 16

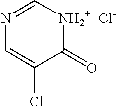

6.5 g (40 mmol) of iron-III-chloride is added to a solution of 77 g (0.8 mol) of 4(3H)-pyrimidinone in 770 ml of glacial acetic acid and 113.6 g (1.6 mol) of chlorine is introduced at 40-45° C. The reaction mixture is cooled to 15° C., the solid product obtained is extracted and washed with ether. 112.5 g (84% of theoretical yield) of 5-chloro-6-oxo-1,6-dihydropyrimidin-1-ium chloride is obtained.

### Preparation of 4(3H)-pyrimidinone

### Example 17

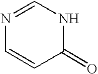

A mixture of 103 g (0.804 mol) of 6-mercapto-4(1H)-pyrimidinone (JP 50053381, Chem. Abstr. CAN 84:17404) and 141.5 g (1.2 mol) of Raney nickel in 1.2 l ethanol is heated for 8 hours under reflux. The solution is hot-filtered, the residue washed with ethanol and the filtrate concentrated at reduced pressure. 67.2 g (87% of theoretical yield) of 4(3H)-pyrimidinone is obtained.

### EXAMPLES OF USAGE

### Example A

Podosphaera test (apple)/protective
Solvent: 24.5 parts by weight of acetone
   24.5 parts by weight of dimethylacetamide
Emulsifier: 1 part by weight of alkyl-aryl-polyglycolether

One part by weight of active substance is mixed with the specified quantities of solvent and emulsifier and the concentrate diluted with water to the desired concentration to prepare a suitable active substance preparation.

To test the protective efficiency, young plants are sprayed with the active substance preparation in the specified amount. After drying the spray coating, the plants are inoculated with an aqueous spore suspension of the apple powdery mildew pathogen Podosphaera leucotricha. The plants are then placed in a greenhouse at about 23° C. and about 70% relative humidity.

The assessment is made 10 days after the inoculation. In this situation 0% means an efficiency corresponding to that of the control whereas an efficiency of 100% means that no attack is observed.

In this test the substances according to the invention given in Examples 1, 2, 3 and 4 show a very high efficiency when used in a quantity of >90%.

### Example B

Venturia Test (apple)/protective
Solvent: 24.5 parts by weight of acetone
   24.5 parts by weight of dimethylacetamide
Emulsifier: 1 part by weight of alkyl-aryl-polyglycolether

One part by weight of active substance is mixed with the specified quantities of solvent and emulsifier and the concentrate diluted with water to the desired concentration to prepare a suitable active substance preparation.

To test the protective efficiency, young plants are sprayed with the active substance preparation in the specified amount. After drying the spray coating, the plants are inoculated with an aqueous conodia suspension of the apple scab pathogen Venturia inaequalis and are then left for 1 day at about 20° C. and 100% relative humidity in an incubation cabinet.

The plants are then placed in a greenhouse at about 21° C. and a relative humidity of about 90%.

The assessment is made 10 days after the inoculation. In this situation 0% means an efficiency corresponding to that of the control whereas an efficiency of 100% means that no attack is observed.

In this test the substances according to the invention given in Example 2 show a very high efficiency when used in a quantity of >90%.

### Example C

In Vitro Test to Determine ED₅₀ for Micro-Organisms
Solvent: Methanol
Emulsifier: Alkylaryl-polyglycolether

2 mg of active substance is mixed with 100 μl of methanol and the concentrate thus produced is then diluted with a mixture of 1000 ml of methanol and 6 g of the afore-mentioned emulsifier to the respectively desired concentration.

10 μl of the preparation in each case is pipetted into the cavities of microtitre plates. After the solvent has evaporated, 200 μl of a potato dextrose medium is added to each cavity after this had previously been mixed with the respectively desired concentration of spores or micelia of the micro-organism to be tested. The resulting concentrations of active substance in the cavities are

| | |
|---|---|
| 0.1 | ppm |
| 1 | ppm |
| 10 | ppm |
| and 100 | ppm. |

The resulting concentration of emulsifier is 300 ppm in each case.

For incubation the microtitre plates are then moved on a shaker for 3 to 5 days at 22° C. until sufficient growth of the respective micro-organism can be detected in the untreated control.

The evaluation is made photometrically at a wavelength of 620 nm. The measurement data for the various concentrations are used to calculate the active substance dose which gives a 50% inhibition of fungal growth (ED₅₀) compared with the untreated control.

In this test the ED₅₀ value of the compounds according to the invention given in Examples 1 and 2 for Botrytis cinerea is obtained at an active substance dose lower than 10 ppm.

1. A compound of the formula 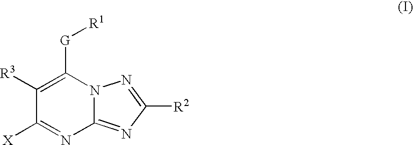
in which
R¹ represents optionally substituted alkyl, optionally substituted alkenyl, optionally substituted alkynyl, optionally substituted cyloalkyl or optionally substituted heterocyclyl,
R² represents a hydrogen, halogen, optionally substituted alkyl or optionally substituted cycloalkyl,
R³ represents optionally substituted heterocyclyl,
G represents oxygen or SOₙ, wherein
n is 0, 1 or 2,
and
X represents halogen, cyano, optionally substituted alkyl, optionally substituted alkoxy, optionally substituted alkylthio, optionally substituted alkylsulphinyl or optionally substituted alkylsulphonyl.

2. A compound of the formula (I) according to claim 1, in which
R¹ represents alkyl with 1 to 6 carbon atoms which can be identically or differently substituted between one and five times, by halogen, cyano, hydroxy, alkoxy with 1 to 4 carbon atoms or cycloalkyl with 3 to 6 carbon atoms, or
R¹ represents alkenyl with 2 to 6 carbon atoms which can be identically or differently substituted between one and three times, by halogen, cyano, hydroxy, alkoxy with 1 to 4 carbon atoms or cycloalkyl with 3 to 6 carbon atoms, or
R¹ represents alkynyl with 3 to 6 carbon atoms which can be identically or differently substituted between one and three times, by halogen, cyano, hydroxy, alkoxy with 1 to 4 carbon atoms or cycloalkyl with 3 to 6 carbon atoms, or
R¹ represents cycloalkyl with 1 to 6 carbon atoms which can be identically or differently substituted between one and three times, by halogen or alkyl with 1 to 4 carbon atoms, or
R¹ represents saturated or unsaturated heterocyclyl with 5 or 6 ring members and 1 to 3 heteroatoms selected from the group consisting of nitrogen, oxygen and sulphur, wherein the heterocyclyl can be substituted once or twice by halogen, alkyl with 1 to 4 carbon atoms, cyano and/or cycloalkyl with 3 to 6 carbon atoms,
R² represents hydrogen, fluorine, chlorine, bromine, iodine, alkyl with 1 to 4 carbon atoms, haloalkyl with 1 to 4 carbon atoms and 1 to 9 halogen atoms or cycloalkyl with 3 to 6 carbon atoms,
R³ represents saturated or unsaturated heterocyclyl with 5 or 6 ring members and 1 to 4 heteroatoms selected from the group consisting of nitrogen, oxygen and sulphur, wherein the heterocyclyl can be identically or differently substituted between one and four times by fluorine, chlorine, bromine, cyano, nitro, alkyl, alkoxy, hydroximinoalkyl or alkoximinoalkyl with respectively 1 to 3 carbon atoms per part alkyl, haloalkyl or haloalkoxy with respectively 1 to 3 carbon atoms and 1 to 7 halogen atoms
G represents oxygen or SOₙ, wherein
n is 0, 1 or 2,
and
X represents fluorine, chlorine, bromine, cyano, alkyl with 1 to 4 carbon atoms, alkoxy with 1 to 4 carbon atoms, alkyl sulphinyl with 1 to 4 carbon atoms or alkyl sulphonyl with 1 to 4 carbon atoms.

3. A compound of formula (I) according to claim 1, in which
R¹ represents a residue of the formula 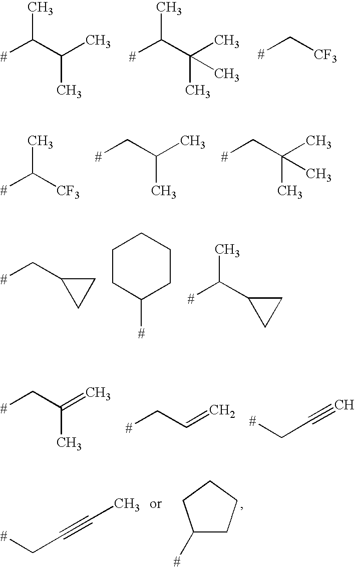
where # marks the linking point,
R² represents hydrogen, fluorine, chlorine, bromine, iodine, methyl, ethyl, isopropyl, cyclopropyl, cyclobutyl, cyclopentyl, cyclohexyl, trifluoromethyl, 1-trifluoromethyl-2,2,2-trifluoroethyl or heptafluorisopropyl,
R³ represents pyridyl which is linked in the 2- or 4-position and can be identically or differently substituted between one and four times by fluorine, chlorine, bromine, cyano, nitro, methyl, ethyl, methoxy, methylthio, hydroximinomethyl, hydroximinoethyl, methoximinomethyl, methoximinoethyl or trifluoromethyl, or
R³ represents pyrimidyl which is linked in the 2- or 4-position and can be identically or differently substituted between one and three times by fluorine, chlorine, bromine, cyano, nitro, methyl, ethyl, methoxy, methylthio, hydroximinomethyl, hydroximinoethyl, methoximinomethyl, methoximinoethyl or trifluoromethyl, or
R³ represents thienyl which is linked in the 2- or 3-position and can be identically or differently substituted between one and three times by fluorine, chlorine, bromine, cyano, nitro, methyl, ethyl, methoxy, methylthio, hydroximinomethyl, hydroximinoethyl, methoximinomethyl, methoximinoethyl or trifluoromethyl, or
R³ represents thiazolyl which is linked in the 2-, 4- or 5-position and can be identically or differently substituted once or twice by fluorine, chlorine, bromine, cyano, nitro, methyl, ethyl, methoxy, methylthio, hydroximinomethyl, hydroximinoethyl, methoximinomethyl, methoximinoethyl and/or trifluoromethyl,
G represents oxygen or sulphur and
X represents fluorine, chlorine, bromine, cyano, methyl, methoxy or methylthio.

4. A method for producing triazolopyrimidines of formula (I) according to claim 1, comprising
(a) reacting a compound of the formula 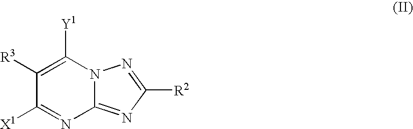
in which
R² and R³ have the meanings given in claim 1,
X¹ represents halogen and
Y1 represents halogen,
with compounds of the formula
R¹-G-H (III)
in which
R¹ and G have the meanings specified in claim 1, optionally in the presence of a diluent, optionally in the presence of an acid acceptor and optionally in the presence of a catalyst and optionally the compound thus obtained of the formula 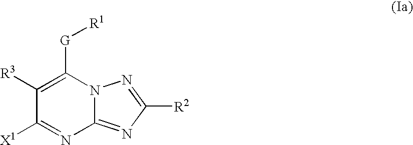
in which
R¹, R², R³, G and X¹ have the meanings specified above,
are either reacted
a) with compounds of the formula
R⁴-Me (IV)
in which
R⁴ represents optionally substituted alkoxy, optionally substituted alkylthio, optionally substituted alkylsulphinyl, optionally substituted alkylsulphonyl or cyano and
Me represents sodium or potassium,
optionally in the presence of a catalyst,
or
b) with compounds of the formula
R⁵—MgHal (V)
in which
R⁵ represents optionally substituted alkyl and
Hal represents chlorine or bromine,
in the presence of a diluent.

5. A composition useful for combating undesirable micro-organisms, comprising at least one compound of formula (I) according to claim 1 in addition to extenders and/or surfactants.

6. (canceled)

7. A method for combating undesirable micro-organisms, comprising contacting one or more compounds of formula (I) according to claim 1 with the undesirable micro-organisms and/or their habitat.

8. A method for preparing the composition of claim 5, comprising contacting one or more said compounds of formula (I) with extenders and/or surfactants.

9. A compound of the formula 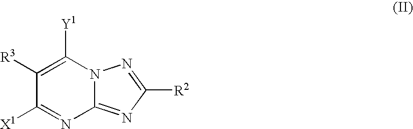
in which
R² represents hydrogen, halogen, optionally substituted alkyl or optionally substituted cycloalkyl,
R³ represents optionally substituted heterocyclyl,
X¹ represents halogen and
Y¹ represents halogen.

10. A method for producing a compound of formula (II) according to claim 9, comprising contacting
(a) a compound of the formula 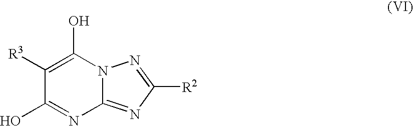
in which
R² and R³ have the meanings given in claim 9,
with halogenating agents, optionally in the presence of a diluent.

11. A compound of the formula 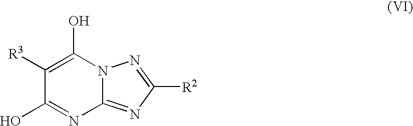
in which
R² represents hydrogen, halogen, optionally substituted alkyl or optionally substituted cycloalkyl and
R³ represents optionally substituted heterocyclyl.

12. A process for preparing a compound of formula (VI) according to claim 11, comprising contacting
(a) a compound of the formula 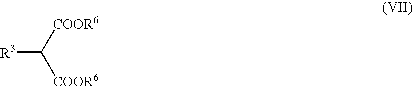
in which
R³ has the meaning specified in claim 11 and
R⁶ represents alkyl with 1 to 4 carbon atoms,
with a compound of the formula 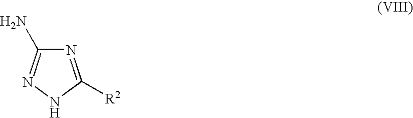
in which
R² has the meaning given in claim 11,
optionally in the presence of a diluent and optionally in the presence of an acid binder.

13. A compound of the formula 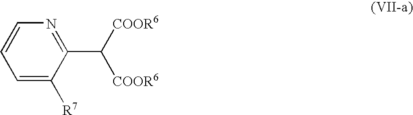
in which
R⁶ represents alkyl with 1 to 4 carbon atoms and
R⁷ represents halogen or haloalkyl.

14. A process for preparing a compound of formula (VII-a) according to claim 13, comprising reacting
(a) a compound of the formula 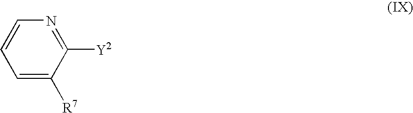
in which
R⁷ has the meaning specified in claim 13 and
Y² represents halogen,
with a compound of the formula 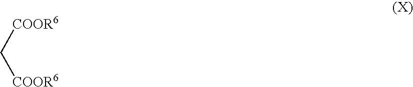
in which
R⁶ has the meaning specified in claim 13, optionally in the presence of a diluent, optionally in the presence of a copper salt and optionally in the presence of an acid acceptor.

15. A compound of the formula 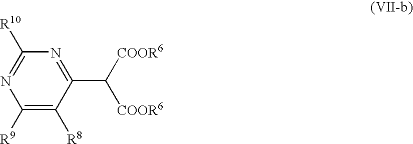
in which
R⁶ represents alkyl with 1 to 4 carbon atoms,
R⁸ represents halogen or haloalkyl and
R⁹ and R¹⁰ independently of one another represent hydrogen, fluorine, chlorine, bromine, methyl, ethyl or methoxy.

16. A process for preparing a compound of formula (VII-b) according to claim 15, comprising reacting
(a) a compound of the formula 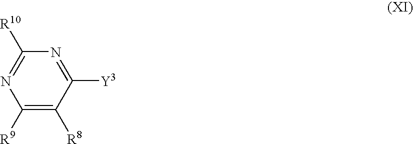
in which
R⁸, R⁹ and R¹⁰ have the meanings specified in claim 15 and
Y³ represents halogen,
with a compound of the formula 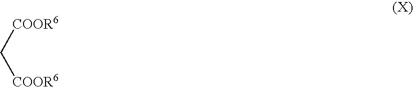
in which
R⁶ has the meaning specified in claim 15, optionally in the presence of a diluent, optionally in the presence of a copper salt and optionally in the presence of an acid acceptor.
